(19) United States
(12) Patent Application Publication
(10) Pub. No.: US 2026/0022316 A1
(43) Pub. Date: Jan. 22, 2026

(54) AN ASSAY PLATE SYSTEM TO ASSAY A CELL CULTURE MODEL

(71) Applicant: HOOKE BIO LTD., Shannon, Co. Clare (IE)

(72) Inventors: Mark LYONS, Shannon (IE); Finola CLIFFE, Shannon (IE); Conor MADDEN, Shannon (IE); Patrick COSTELLO, Shannon (IE); Shane DEVITT, Shannon (IE); Sumir RAMESH MUKKUNDA, Shannon (IE); Bhairavi BENGALURU KESHAVA, Shannon (IE)

(73) Assignee: HOOKE BIO LTD., Shannon (IE)

(21) Appl. No.: 18/993,490

(22) PCT Filed: Jul. 13, 2023
(86) PCT No.: PCT/EP2023/069485

§ 371 Date: Jan. 10, 2025

(30) Foreign Application Priority Data
Jul. 13, 2022 (EP) 22184823.7
Aug. 24, 2022 (EP) 22191885.7

(51) Int. Cl. C12M 1/32; C12M 1/00; C12M 1/34

(52) U.S. Cl. CPC C12M 23/12; C12M 23/22; C12M 23/38; C12M 23/40; C12M 29/10; C12M 29/14; C12M 41/36

(57) ABSTRACT

An assay plate system for assaying a cell culture model, such as an organoid, comprises an assay plate, an upper surface, a grid array of wells comprising at least two rows of wells, each row comprising at least two wells, each well having an open top formed in the upper surface and a closed transparent base, and at least two columns of wells, each column comprising at least three wells, each well having an open top formed in the upper surface and a closed transparent base. A first gasket comprising a plate having a top surface and a bottom surface comprising a plurality of elongated recesses is provided that together with the upper surface of the assay plate define a fluidic conduit for each row of wells providing fluidic communication between the first fluid inlet aperture, the row of wells and the first fluid outlet aperture in series.

### FIELD OF THE INVENTION

The present invention relates to an assay plate system to assay a cell culture model and especially a 3-D cell culture model such as an organoid or microtissue. Also contemplated is a method of assaying a cell culture model that employs an assay plate system of the invention.

### BACKGROUND TO THE INVENTION

During the process of drug discovery, test drugs need to be tested on cells to determine how they work and the effects of the drug on the cells. To date, the pharmaceutical industry has relied primarily on animal models and human cell line cultures that bear little resemblance to normal or disease human tissue, resulting in only one in every ten drugs making it through clinical testing. This high failure rate in clinical trials of drugs that make it through pre-clinical testing adds greatly to the cost of drug development.

Microtissues or organoids are tiny, self-organized three-dimensional tissue cultures that are derived from stem cells and other cell types such as primary cells. Such cultures can be crafted to replicate much of the complexity of an organ, or to express selected aspects of it like producing only certain types of cells. Organoids/microtissues may be grown from a variety of precursor cell types including stem cells—cells that can divide indefinitely and produce different types of cells as part of their progeny. Scientists have learned how to create the right environment for the precursor cells so they can follow their own genetic instructions to self-organize, forming tiny structures that resemble miniature organs composed of many cell types. Organoids/microtissues can range in size from less than the width of a hair to 5 mm.

Many scientists believe that microtissues have the capacity to be more accurate and physiologically relevant models than existing animal models and that using organoids in pre-clinical testing of drugs will reduce the failure rate of drugs that enter clinical trials. These models will allow drug and vaccine manufacturers to bring products to market in a more timely and efficient manner. There is therefore a need for a technology to facilitate high throughput organoid screening that reproduces physiological conditions (e.g., temperature, CO2 and nutrients), facilitates fluid flow to the organoids, and allows organoids to be imaged.

It is an objective of the invention to overcome at least one of the above-referenced problems.

### SUMMARY OF THE INVENTION

The applicant provides an assay plate system suitable for assaying in real-time cell culture models, especially 3-D cell culture models such as organoids, microtissues and spheroids, generally in a high-throughput manner. The system comprises a disposable assay plate containing one or more fluidic conduits, each having at least two or three wells, a fluid inlet conduit and a fluid outlet conduit. The wells are formed in an upper surface of an assay plate and have transparent bases allowing well contents to be imaged, and the conduits are defined by the upper surface of the assay plate, and a gasket that overlies the plate to form conduits in the plate comprising rows or columns of wells. The gasket can be a unitary plate having a top surface at least a part of which is gas-permeable (to allow gases diffuse into the conduits) and a bottom part having recesses configured to define the conduits (FIGS. 26-29) or may comprise a flexible film placed over the assay plate, and a spacer disposed between the film and assay plate (FIGS. 1-9). Making a unitary plate gasket from Teflon has been found to be advantageous as the Teflon allows the gasket to be machined (making manufacture easier), the Teflon does not interact with or adsorb with molecules on the plate, and is stiff enough to allow easy handling when the gasket is adjusted or replaced. The assay plate can be used to determine the effects of an agent such as a drug on different organoids, and also to test if the effects of the drug on one organoid (say for example a heart organoid) have an effect on a second organoid (say for example a liver organoid). In one aspect, the assay plate system of the invention includes a first gasket that defines conduits containing the rows of wells, and a second gasket that defines conduits containing the columns of wells. The system may be initially assembled using the second gasket so that conduits comprising columns of wells are formed on the plate (e.g. for use when cell culture models are being grown or matured in-situ in the wells), and the second gasket is then removed and replaced with the first gasket that defines conduits comprising columns of wells (e.g. for use when the effects of a test molecule on the cell culture models are being assayed). When the grid array of wells has the same number of columns and rows, the same gasket may be used simply by rotating the gasket relative to the assay play by 90 degrees.

In one aspect, the assay plate system also includes a fluidics system to allow a fluid to be passed along columns of wells, and then when the gasket has been replaced (or repositioned), to be passed along rows of wells. This is enabled by providing an assay plate having a fluid inlet conduit and fluid outlet conduit at opposite ends of the rows of well and the columns of wells. The conduits pass through the assay plate and are configured to fluidically communicate with fluidic conduits provided in a base plate that receives the assay plate when the system is assembled. In addition, the gasket and cover plates may include apertures to allow fluid in any conduit to be circulated during an assay. In practice, this allows molecules that are released by one organoid of one tissue type to be exposed to an organoid of a second tissue type.

In a first aspect, there is provided an assay plate system for assaying a cell culture model, the assay plate system comprising
an assay plate comprising an upper surface and comprising a grid array of wells comprising:
at least two rows of wells, each well having an open top formed in the upper surface and a closed transparent base;
at least two columns of wells, each well having an open top formed in the upper surface and a closed transparent base;
a fluid inlet conduit for each row of wells having a first fluid inlet aperture disposed on the upper surface at a first end of each row of wells; and
a fluid outlet conduit for each row of wells having a first fluid outlet aperture disposed on the upper surface at a second end of each row of wells.

The assay plate system typically comprises a first gasket comprising a plate having a top surface, a bottom surface, and a plurality of elongated recesses formed in the bottom surface that together with the upper surface of the assay plate define a fluidic conduit for each row of wells providing fluidic communication between the first fluid inlet aperture, the row of wells and the first fluid outlet aperture in series.

The assay plate system typically comprises a cover plate configured to abut the top part of the gasket upon assembly of the assay plate system.

In any embodiment, the gasket is formed from Teflon. The use of Teflon for the gasket has been found to be advantageous as it can be machined to allow for more accurate manufacture, it does not interact with or adsorb molecules in the plate, and it is a stiffer material that silicone allow the gasket to be more easily handled.

In any embodiment, the assay plate system comprises a second gasket configured for replacement of the first gasket in the assembled assay plate system, the second gasket comprising a plate having a top surface, a bottom surface, and a plurality of elongated recesses formed in the bottom surface that together with the upper surface of the assay plate define a fluidic conduit for each column of wells providing fluidic communication between the column of wells.

The provision of an assay plate system having a modular first and second gaskets allows the system to be first assembled to provide fluidic conduits on the top of the assay plate containing columns of wells before the assay plate is reassembled with the second gasket to provide fluidic conduits on the top of the assay plate containing rows of wells. As explained in further detail below, this allows the assay plate to be assembled in a cell growth/maturation configuration and then reassembled in a microtissue assay configuration.

In any embodiment, the top wall of the recesses (which form the top wall of the conduits when the system is assembled) are gas permeable and ideally liquid impermeable. The top of the recess typically has a thickness of 1 to 2 mm, 1.4 to 1.8 mm, or about 1.6 mm. When the gasket is formed from Teflon, a wall thickness of about 1.6 mm is sufficiently thin to allow gas diffusion through the wall.

In any embodiment, the sidewalls of the recesses (which form the sides of the conduits when the system is assembled) have a height of 1 to 3 mm, 1.5 to 2.5 mm, or about 2.0 mm.

In any embodiment, the assay plate system comprises a first gasket configured to form conduits comprising rows of wells and a second gasket configured to form conduits comprising rows of wells. In any embodiment, the assay plate has a grid array of assay wells where the number of columns is different to the number of rows, wherein

In another embodiment, the grid array of wells has the same number of columns and rows of wells.

In any embodiment, the assay plate comprises:
a fluid inlet conduit for each column of wells having a first fluid inlet aperture disposed on the upper surface at a first end of each column of wells; and
a fluid outlet conduit for each column of wells having a first fluid outlet aperture disposed on the upper surface at a second end of each column of wells.

The provision of a fluid inlet and outlet conduits at each end of a column of wells allows fluid to be directed along the columns of wells (for example, when growing cells into organoids).

In any embodiment, the fluid inlet conduit for each row or column of wells comprises a second fluid inlet aperture disposed on a lower surface of the assay plate at the first end of each row or column of wells.

In any embodiment, the fluid outlet conduit for each row or column of wells comprise a second fluid outlet aperture disposed on a lower surface of the assay plate at the second end of each row or column of wells.

The arrangement of the fluid inlet and outlet conduits allows assay fluids to be provided to the columns or rows of wells from underneath the plate.

In any embodiment, the assay plate system comprises a base plate comprising a central through aperture comprising a recessed shoulder that extends around a periphery of the central through aperture, in which the recessed shoulder is dimensioned to receive the assay plate. This provides an effective base for receiving the assay plate and gasket plate and retaining them in position during the assay.

In any embodiment, the base plate comprises first fluidic conduits configured to fluidically connect with the second fluid inlet apertures of the assay plate when the assay plate is mounted on the recessed shoulder of the base plate.

In any embodiment, the base plate comprises second fluidic conduits configured to fluidically connect with the second fluid outlet apertures of the assay plate when the assay plate is mounted in the recessed shoulder of the base plate.

The provision of first and second fluidic conduits in the base plate allow the assay plate to be nested securely within the base plate while allowing the fluidics supply to the assay plate to be routed through the base plate.

In any embodiment, the fluid inlet conduits and fluid outlet conduits of the assay plate comprise extension sections that extend proud of a lower surface of the assay plate, and in which the recessed shoulder of the base plate comprises sockets dimensioned to receive the extension sections and fluidically connect the fluid inlet conduits and fluid outlet conduits of the assay plate with the first fluidic conduits and second fluidic conduits of the base plate, respectively.

In any embodiment, the first fluidic conduit and second fluidic conduit of the base plate comprises an outlet/inlet aperture disposed on an external sidewall of the base plate.

In any embodiment, an upper surface of the base plate is configured for coupling to the cover plate such that when the base plate and cover plate are coupled together with the assay plate and gasket sandwiched between the base plate and cover plate, the cover plate abuts the top of the gasket plate.

In any embodiment, the recessed shoulder of the base plate is configured to allow the assay plate and gasket nest within the base plate.

In any embodiment, assay plate system comprises a first fluid supply system to supply fluid to at least a first row of wells, the first fluid supply system comprising a first fluidic supply inlet conduit fluidically connected to an inlet aperture of a first fluidic conduit of the base plate, a first fluidic supply outlet conduit fluidically connected to an outlet aperture of a second fluidic conduit of the base plate, and a pump operable to pump fluid along the first row of wells.

In any embodiment, assay plate system comprises a dedicated first fluid supply system for each row of wells,

In any embodiment, assay plate system comprises a second fluid supply system to supply fluid to at least a first column of wells, the second fluid supply system comprising a second fluidic supply inlet conduit fluidically connected to an inlet aperture of a first fluidic conduit of the base plate, a second fluidic supply outlet conduit fluidically connected to an outlet aperture of a second fluidic conduit of the base plate, and a pump operable to pump fluid along the first column of wells.

In any embodiment, assay plate system comprises a dedicated second fluid supply system for each column of wells,

In any embodiment, at least one of the elongated recesses in the gasket comprises an aperture formed at each end of the elongated recess, and the cover plate comprises corresponding apertures configured to register with the apertures in the gasket when the assay plate system is assembled.

This provides a fluid recirculation system for each conduit, and in practice is employed when the organoids are being assayed to allow molecules produced by an organoid of one tissue type to be incubated with an organoid of a second tissue type.

In any embodiment, assay plate system comprises a fluid recirculation system to recirculate fluid across wells in a row of wells, comprising a fluid recirculation conduit having a first end in fluid communication with a first aperture in the cover plate and a second end in fluid communication with an opposed second aperture in the cover plate, and a pump operable to pump fluid along the fluid recirculation conduit.

In any embodiment, each elongated recesses in the gasket comprises an aperture formed at each end of the elongated recess, the cover plate comprises corresponding apertures configured to register with the apertures in the gasket when the assay plate system is assembled, wherein the assay system comprises a fluid recirculation system for each row of wells.

In any embodiment, the assay plate has a peripheral sidewall defining a recessed upper surface of the plate, wherein the gasket is dimensioned to nest within recessed upper surface of the plate.

In another embodiment, the gasket is provided by a flexible film that is gas permeable and liquid impermeable and dimensioned to cover the at least one assay well system, and a spacer element configured to space the flexible film from the upper surface of the assay plate over the assay well system to form a fluidic conduit providing fluidic communication between the fluid inlet aperture, the row of wells and the fluid outlet aperture in series.

In any embodiment, the assay plate comprises at least 2, 3, 4, 5, 6, 7 or 8 rows of wells.

In any embodiment, the assay plate comprises at least 2, 3, 4, 5, 6, 7 or 8 columns of wells.

In any embodiment, the spacer element comprises one or more projections that extend upwardly from the upper surface of the assay plate. Generally, the or each projection is disposed adjacent each well. Generally, the cover plate is configured to abut the flexible film to form part or all of a periphery of the or each fluidic conduit.

In any embodiment, the spacer element comprises a first projection disposed along a longitudinal axis of the row of wells and a second projection spaced apart from the first projection around the well.

In any embodiment, the spacer element comprises (as an alternative, or in addition, to the projection(s)) a gasket plate configured to sit between the flexible film and the upper surface of the assay plate when the assay plate system is assembled, in which the gasket comprises one or more fluidic conduit shaped aperture. Thus, when the assay plate comprises three assay well systems, the gasket will generally comprise three fluidic conduit shaped apertures.

In any embodiment, the assay plate system comprises a base plate to receive and hold the cover plate.

In any embodiment, the base plate comprises a first fluidic conduit configured to fluidically connect with the fluid inlet conduit of the assay plate when the assay plate is received in the base plate.

In any embodiment, the base plate comprises a second fluidic conduit configured to fluidically connect with the fluid outlet conduit of the assay plate when the assay plate is received in the base plate.

In any embodiment, the base plate comprises at least one central through aperture comprising a recessed shoulder (that generally extends around a periphery of the central through aperture), in which the recessed shoulder is dimensioned to receive the assay plate.

In any embodiment, the assay plate comprises a plurality of assay well systems, and wherein the base plate comprises a first fluidic conduits and a second fluidic conduit corresponding to each assay well system.

In any embodiment, the fluid inlet conduits and fluid outlet conduits extend proud of a lower surface of the assay plate, and in which the recessed shoulder of the base plate comprises a first socket dimensioned to receive the extended fluid inlet conduit and fluidically connect the fluid inlet conduit with the first fluidic conduit of the base plate, and in which the recessed shoulder of the base plate comprises a second socket dimensioned to receive the extended fluid outlet conduit and fluidically connect the fluid outlet conduit with the second fluidic conduit of the base plate.

In any embodiment, each first fluidic conduit and second fluidic conduit of the base plate comprises an outlet disposed in a sidewall of the base plate.

In any embodiment, the upper surface of the base plate is configured for coupling to the cover plate such that when the base plate and cover plate are coupled together with the assay plate and flexible film sandwiched between the base plate and cover plate, the cover plate abuts the flexible gas-impermeable film.

In any embodiment, the flexible film comprises an aperture corresponding to each fluid inlet aperture and fluid outlet aperture of the assay plate.

In any embodiment, the cover plate comprises:
a cover plate inlet conduit configured to fluidically connect with the fluidic conduit of the assay plate adjacent to the fluid inlet aperture of the assay plate; and
a cover plate outlet conduit configured to fluidically connect with the fluidic conduit of the assay plate adjacent to the fluid outlet aperture of the assay plate.

In any embodiment, the assay plate comprises a plurality of fluidic conduits, and wherein the cover plate comprises a cover plate inlet conduit and cover plate outlet conduit corresponding to each fluidic conduit.

In any embodiment, the assay plate system comprises a pump fluidically connected to each pair of cover plate inlet conduit and cover plate outlet conduit and configured to recirculate fluid through the fluidic conduit of the assay plate.

In any embodiment, the assay plate comprises a plurality of assay well systems each having a fluidic conduit, in which the system comprises a separate pump for each fluidic conduit.

In any embodiment, the assay plate system comprises a fluid supply conduit fluidically connected to the first fluidic conduit of the base plate and a fluid removal conduit fluidically connected to the second fluidic conduit of the base plate.

In any embodiment, the assay plate comprises a n×m array of wells, where n and m are each independently a number from 2 to 500, 2 to 100, 2-50, 2-20 or 2-10.

In any embodiment, the assay plate comprises an array of wells comprising:
at least two rows of wells that define a plurality of columns of wells;
second fluid inlet conduits having a second fluid inlet aperture disposed on the upper surface of the assay plate at a first end each of the columns of wells; and
second fluid outlet conduits having a second fluid outlet aperture disposed on the upper surface of the assay plate at a second end each of the columns of wells.

The second fluid inlet and outlet conduits provide for fluid flow across a column of wells. Fluid flow in this direction may be employed during a stage of microtissue maturation or during maintenance of the microtissue. The flow can be then switched to the (first) fluid inlet and outlet conduits for fluid flow along the rows of wells

In any embodiment, the assay plate system comprises a second spacer element configured to space the flexible film from the upper surface of the assay plate over the assay well system to form a fluidic conduit providing fluidic communication between the second fluid inlet aperture, the columns of wells and the second fluid outlet aperture in series.

In any embodiment, the second spacer element comprises a gasket plate configured to sit between the flexible film and the upper surface of the assay plate when the assay plate system is assembled, in which the gasket comprises one or more fluidic conduit shaped aperture. Thus, when the assay plate comprises three assay well systems, the gasket will generally comprise three fluidic conduit shaped apertures.

In any embodiment, the assay plate system additionally comprises a support plate, a plurality of valve block manifolds, a well perfusion control valves module associated with each valve block manifold, and fluidic conduits fluidically connecting the valve block manifolds in a fluid circuit. An example of the system is shown in FIG. 24.

In another aspect, the invention provides a method of assaying a cell culture model (for example a 3-D cell culture model such as an organoid) that employs an assay plate system of the invention, comprising the steps of:
placing a first cell culture model in a first well of a first assay well system;
delivering a fluid comprising a test molecule to the fluidic conduit of the first assay well system so that the test molecule is incubated with the first cell culture model; and
imaging the first cell culture model through the transparent base of the first well with an imaging device.

In another aspect, there is provided a method of assaying a microtissue that employs an assay plate system of the invention, comprising the steps of:
placing a first microtissue sample in a first well of a first row of wells;
placing a second microtissue sample in a second well of the first row of wells;
optionally, placing a third microtissue sample in a third well of the first row of wells;
delivering a first fluid to the fluid inlet conduit of the first row of wells so that the first fluid is incubated with each microtissue sample in the first row of wells; and
imaging the wells through the transparent base of each well with an imaging device.

In any embodiment, the method comprises the steps of:
placing a first microtissue sample in a first well of a second row of wells;
placing a second microtissue sample in a second well of the second row of wells;
optionally, placing a third microtissue sample in a third well of the second row of wells;
delivering a second fluid to the fluid inlet conduit of the second row of wells so that the second fluid is incubated with each microtissue sample in the second row of wells; and
imaging the wells of the second row of wells through the transparent base of each well with an imaging device.

In any embodiment, the method comprises the step of recirculating fluid across the wells or each row of wells.

In any embodiment, the first fluid comprises a first concentration of a first test molecule and the second fluid comprises a second concentration of the first test molecule.

In any embodiment, the first fluid comprises a first test molecule and the second fluid comprises a second test molecule.

In another aspect, there is provided a method of assaying a microtissue that employs an assay plate system of the invention, comprising the steps of:
seeding cells in the wells of a first column of wells;
seeding cells in the wells of a second column of wells;
assembling the assay plate system such that the gasket and assay plate define a first conduit comprising the first column of wells and a second conduit comprising the second column of wells;
delivering a first fluid to the first column of wells in the first conduit to mature the cells in the wells to form microtissues;
delivering a second fluid to the second column of wells in the second conduit to mature the cells in the wells to form microtissues;
reassembling the assay plate system such that the gasket and assay plate define a third conduit comprising a first row of wells and a fourth conduit comprising a second row of wells;
delivering a third fluid to the first row of wells in third conduit; and
delivering a fourth fluid to the second row of wells in the fourth conduit; and
imaging the wells of the first and second row of wells through the transparent base of each well with an imaging device.

In any embodiment, the step of reassembling the assay plate system comprises replacing the gasket with a gasket configured to provide conduits comprising rows of wells.

In any embodiment, the cells seeded into the wells are stem cells, primary cells or cell lines, in which the first fluid comprises a chemical configured to differentiate the stem cells into a first microtissue type and the second fluid comprises a chemical configured to differentiate the stem cells into a second microtissue type.

In any embodiment, the grid array of wells of the assay plate comprises the same number of columns and rows of wells, in which the step of reassembling the assay plate system comprises reorienting the same gasket plate.

In any embodiment, the first and second fluids comprises a growth media and the third and fourth fluids each, independently, comprise a test molecule.

In any embodiment, the method comprises delivering an assay fluid comprising a diagnostic reagent to the wells of the assay plate prior to the imaging step.

In any embodiment, the method comprises a step of re-circulating assay fluid around the wells in series.

In any embodiment, the method is performed inside a fluid tight environmental chamber. In any embodiment, the method comprises changing the fluid (e.g. gas) content of the environmental chamber during the method. In any embodiment, the method is performed in a first gas environment in the environmental chamber, the gas environment is then modified to the second gas environment, and the method is then performed in the changed gas environment in the environmental chamber

In any embodiment, the method comprises the initial steps of:
placing a cell culture model or cell culture model pre-cursor into the wells of a column of wells;
directing liquid across the wells of the column for maturation of the cell culture model.

In any embodiment, the method comprises the initial steps of:
placing a first cell culture model or cell culture model precursor into the wells of a first column of wells; placing a second cell culture model or cell culture model precursor into the wells of a second column of wells; and
directing liquid across the wells of each column for maturation or maintenance of the first and second cell culture models.

In another aspect, the invention provides an assay rig comprising a frame, an assay plate system of the invention attached to the frame, an imaging device attached to the frame and disposed underneath the assay plate system when the assay plate system is mounted to the holder.

In any embodiment, the rig comprises a coupling device for coupling the imaging device to the frame, wherein the coupling device is configured to move the imaging device relative to the holder so as to allow different wells of the assay plate to be imaged.

In any embodiment, the rig comprises a processor operatively coupled to the coupling device and the imaging device and configured to generate images of specific wells in response to input instructions.

In any embodiment, the rig is contained within an environmental chamber. In any embodiment, the environmental chamber is configured to modify one or more environmental parameters inside the chamber, for example a parameter selected from temperature, O₂ level, CO₂ level, and moisture level.

There is also disclosed an assay plate system for assaying a cell culture model, the assay plate system comprising:
an assay plate comprising an upper surface and comprising a grid array of wells comprising:
   at least two rows of wells, each row of wells comprising at least two or three wells, each well having an open top formed in the upper surface and a closed transparent base;
   a fluid inlet conduit for each row of wells having a first fluid inlet aperture disposed on the upper surface at a first end of each row of wells;
   a fluid outlet conduit for each row of wells having a first fluid outlet aperture disposed on the upper surface at a second end of each row of wells;
   at least two columns of wells, each column of wells comprising at least two or three wells, each well having an open top formed in the upper surface and a closed transparent base;
   a fluid inlet conduit for each column of wells having a first fluid inlet aperture disposed on the upper surface at a first end of each column of wells; and
   a fluid outlet conduit for each column of wells having a first fluid outlet aperture disposed on the upper surface at a second end of each column of wells.
a gasket configured to abut the upper surface of the assay plate and define, together with the upper surface of the assay plate, a fluidic conduit for each row of wells providing fluidic communication between the first fluid inlet aperture, the row of wells and the first fluid outlet aperture in series; and
optionally, a cover plate configured to abut a top part of the gasket upon assembly of the assay plate system.

There is also disclosed an assay plate system for assaying a cell culture model, the assay plate system comprising:
an assay plate comprising an upper surface and comprising a grid array of wells comprising:
   at least two rows of wells, each row of wells comprising at least two or three wells, each well having an open top formed in the upper surface and a closed transparent base;
   a fluid inlet conduit for each row of wells having a first fluid inlet aperture disposed on the upper surface at a first end of each row of wells;
   a fluid outlet conduit for each row of wells having a first fluid outlet aperture disposed on the upper surface at a second end of each row of wells;
   at least two columns of wells, each column of wells comprising at least two or three wells, each well having an open top formed in the upper surface and a closed transparent base;
a first gasket configured to abut the upper surface of the assay plate and define, together with the upper surface of the assay plate, a fluidic conduit for each row of wells providing fluidic communication between the first fluid inlet aperture, the row of wells and the first fluid outlet aperture in series;
a second gasket configured to abut the upper surface of the assay plate and define, together with the upper surface of the assay plate, a fluidic conduit for each column of wells providing fluidic communication between the first fluid inlet aperture, the column of wells and the first fluid outlet aperture in series;
optionally, a cover plate configured to abut a top part of the gasket upon assembly of the assay plate system.

There is also disclosed an assay plate system for assaying a cell culture model, the assay plate system comprising:
an assay plate comprising an upper surface and comprising a grid array of wells comprising:
   at least two rows of wells, each row of wells comprising at least two or three wells, each well having an open top formed in the upper surface and a closed transparent base;
   a fluid inlet conduit for each row of wells having a first fluid inlet aperture disposed on the upper surface at a first end of each row of wells;
   a fluid outlet conduit for each row of wells having a first fluid outlet aperture disposed on the upper surface at a second end of each row of wells;
   at least two columns of wells, each column of wells comprising at least two or three wells, each well having an open top formed in the upper surface and a closed transparent base;
a first gasket configured to abut the upper surface of the assay plate and define, together with the upper surface of the assay plate, an assay conduit for each row of wells providing fluidic communication between the first fluid inlet aperture, the row of wells and the first fluid outlet aperture in series; and
optionally, a cover plate configured to abut a top part of the gasket upon assembly of the assay plate system,
wherein the assay plate system comprises a closed fluid recirculation system to recirculate fluid from one end of the assay conduit to an opposite end of the assay conduit. Typically, the fluid recirculation system comprises a fluid recirculation conduit having a first end that fluidically connects with one end of the assay conduit through a first aperture in the gasket, a second end that fluidically connects with the opposite end of the assay conduit through a second aperture in the gasket, and a fluid recirculation pump operable to pump fluid in a circuit comprising the fluid recirculation conduit and the assay conduit.

Other aspects and preferred embodiments of the invention are defined and described in the other claims set out below.

### BRIEF DESCRIPTION OF THE FIGURES

FIG. 1 is an exploded perspective view of an assay plate system of the invention.

FIG. 2 is an exploded elevational view of an assay plate system of the invention.

FIG. 3 is a top plan view of an assay plate forming part of the assay plate system of the invention.

FIG. 4 is a side elevational view of the assay plate of FIG. 4.

FIG. 5 is a perspective view from above of the assay plate of FIG. 3.

FIG. 6 is a top plan view of a cover plate forming part of the assay plate system of the invention.

FIG. 7 is a side elevational view of the cover plate of FIG. 6.

FIG. 8 is a perspective view from under the cover plate of FIG. 6.

FIG. 9 is a top plan view of a base plate forming part of the assay plate system of the invention.

FIG. 10 is a side elevational view of the base plate of FIG. 9.

FIG. 11 is a perspective view from above of the base plate of FIG. 9.

FIG. 12A is a perspective view of the assay plate system of FIG. 9 in an assembled form.

FIG. 12B is a top plan view of the assay plate system of FIG. 9 in an assembled form.

FIG. 12C is an end elevational view of the assay plate system of FIG. 9 in an assembled form.

FIG. 13 is an exploded perspective view of another assay plate system of the invention including assay fluid supply conduits and fluid recirculation conduits and pumps

FIG. 14 is a plan view of the assay plate system of FIG. 13.

FIG. 15 is a sectional side view of the assay plate system of FIG. 14 taken along the lines A-A of FIG. 14.

FIG. 16 is a detailed view of part of the assay plate system shown in FIG. 15.

FIG. 17 is a plan view of the assay plate system of FIG. 13.

FIG. 18 is a sectional side view of the assay plate system of FIG. 17 taken along the lines B-B of FIG. 17.

FIG. 19 is a detailed view of part of the assay plate system shown in FIG. 18.

FIG. 20 is a perspective view of another assay plate system according to the invention in an assembled form.

FIG. 21 is an exploded perspective view of the assay plate system of FIG. 20.

FIG. 22 is a plan view of the assay plate system of FIG. 20.

FIG. 23: (A) Microtissue preparation and transfer to Bioplate timeline, (B) Showing media flow for microtissue maturation, (C) Layout of the Bioplate 4×10 design showing media flow direction for intertissue communication via recirculation perfusion. Organ images reproduced with permission from Flaticon.com.

FIG. 24 Assembly of the assay plate system of the invention. (A) valve block manifold, (B) fluidic fittings, (C) perfusion control valves, (D) base plate, (E) recirculation lid, (F) recirculation pump, (G) support plate.

FIG. 25A is a bottom plan view of a first gasket plate forming part of the assay plate system of the invention.

FIG. 25B is a side elevational of the gasket plate of FIG. 25A.

FIG. 26A is a bottom plan view of a second gasket plate forming part of the assay plate system of the invention.

FIG. 26B is a side view of the gasket plate of FIG. 26A.

FIG. 27 is an exploded perspective view from below of another assay plate system of the invention including the second gasket plate of FIG. 26.

FIG. 28 is an exploded perspective view from above of the assay plate system of the invention including the gasket plate of FIG. 26.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The cell culture model may be a 2-D or 3-D cell culture model. As used herein, the term “3-D cell culture model” refers to a miniaturized and simplified version of an organ produced in vitro in three dimensions that shows realistic micro-anatomy. Examples include organoids, microtissues and spheroids. Organoids/microtissues are derived from one or a few cells from a tissue, embryonic stem cells or induced pluripotent stem cells, which can self-organize in three-dimensional culture owing to their self-renewal and differentiation capacities. Such 3-D cell culture models typically have a maximum dimension of 50-1500 μm. Examples of 3-D cell culture model include liver, hepatic, pancreatic, epithelial, kidney, cardiac, retinal, blastoid, glioblastoma, thyroid and testicular organoids.

As used herein, the term “3-D cell culture model precursor” or “precursor” refers to a cell or composition of cells capable of being cultured to form a 3-D cell culture model. The precursor may be a cell from a specific tissue, a cell line, a patient cell or tissue sample, an embryonic stem cell, or an induced pluripotent stem cell. The assay plate system of the invention may be used to assay cell culture models such as organoids and to grow 2-D or 3-D cell culture model from precursor cells. Growth generally comprises placing a suitable precursor cell type(s) into a well, adding assay fluid (which may be cell culture fluid containing agents to promote the growth of the desired 2-D or 3-D cell culture model), and recirculation of the assay fluid to the well or wells.

As used herein, the term “assay plate system” refers to an assay plate body having assay wells suitable for assaying a 2-D or 3-D cell culture model such as an organoid. The plate has at least two rows of wells each row comprising at least two or three assay wells and generally a fluid inlet conduit and fluid outlet conduit. The plate includes a gasket to form conduits containing rows or columns of wells providing fluidic connection between the fluid inlet conduit, the wells, and the fluid outlet conduit in series, for example a unitary gasket (e.g. a Teflon gasket) or a flexible film to cover the assay well system and a spacer configured to space the film from the top of the assay plate over the wells and apertures of the conduits to form a fluidic conduit This allows an assay liquid to be supplied to the each well. The plate is generally planar and the wells are generally formed in the top of the plate. The wells of the assay plate body generally have a base formed of a light transparent material that allows the wells to be imaged with an imaging system disposed under the plate. The wells are generally U-bottom wells.

The assay plate and spacer/gasket of the system of the invention are generally disposable, whereas the other parts of the system may be re-usable.

As used herein, the term “light transparent material” refers to a material that is transparent. The base of the wells is generally formed from a light transparent material allow the organoids/microtissue in the wells to be imaged with an imaging device from below the second plate. The light transparent material is generally a polymeric material suitable for melting and casting. The light transparent material may be PDMS, cyclic olefin copolymer (COC), perfluoropolyethers (PFPEs), polyurethane, Flexdym, polylactic acid (PLA).

As used herein, the term “well imaging system” refers to an imaging device configured to image the wells of the plate assembly, generally from a position beneath the plate assembly. Examples of imaging devices include microscopes that can undertake white light and fluorescent imaging.

As used herein, the term “Teflon” refers to polytetrafluoroethylene (PTFE), a synthetic fluoropolymer of tetrafluoroethylene, and similar polymers that are liquid impermeable, gas permeable, and machinable, for example, fluorinated ethylene propylene, polychlorotrifluoroethylene, Perfluoroalkoxy alkane (PFA) and Fluorinated ethylene propylene (FEP). The term “machining” has its art-recognised meaning, e.g. to reduce or finish an article by turning, shaping, planing, or milling by machine-operated tools.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

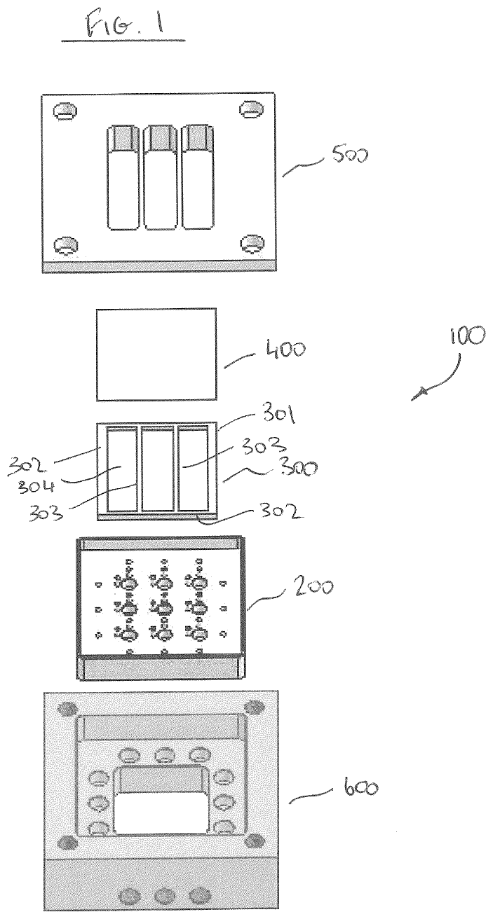

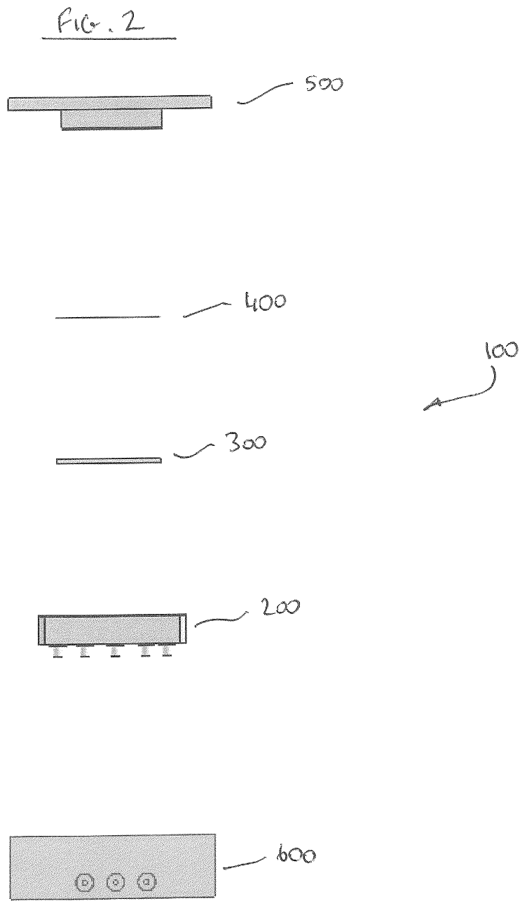

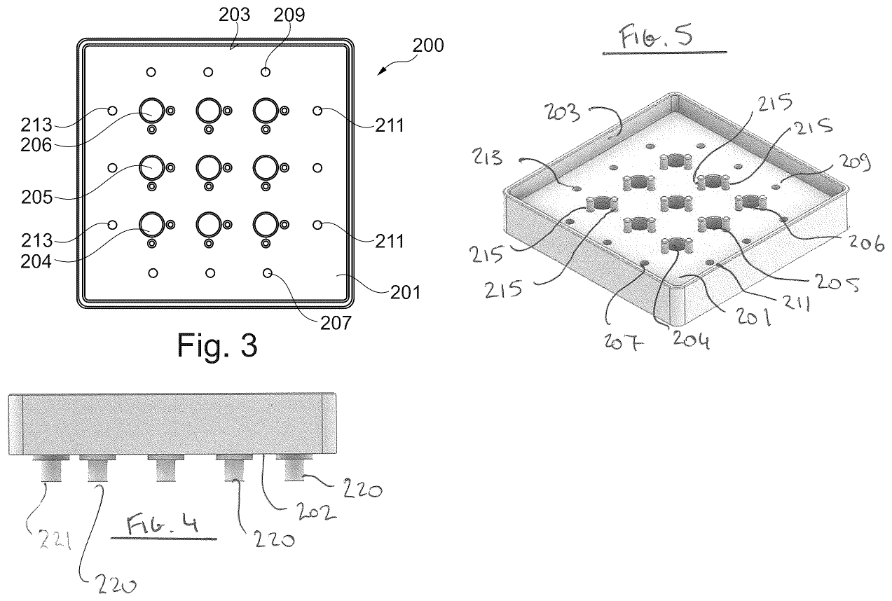

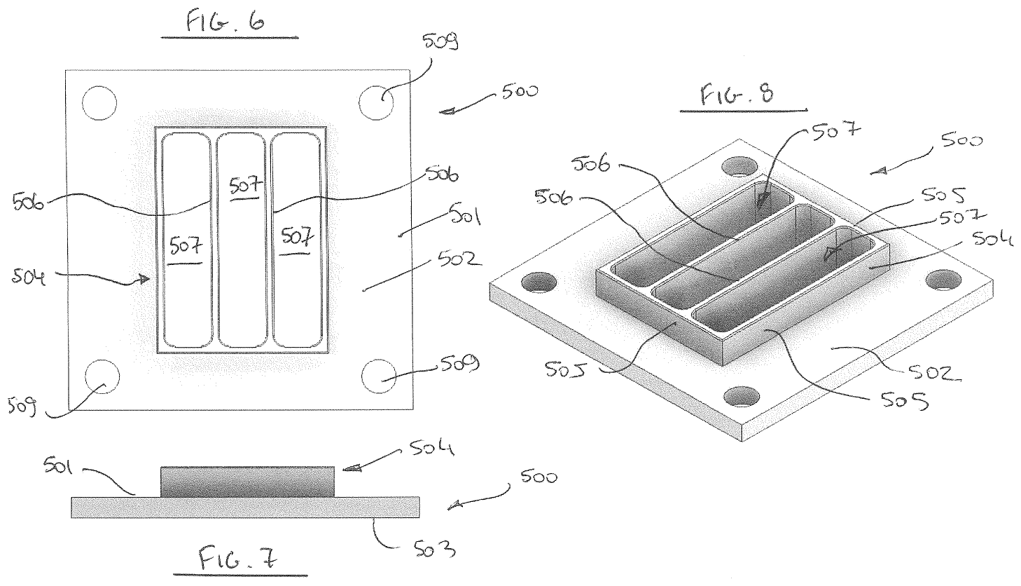

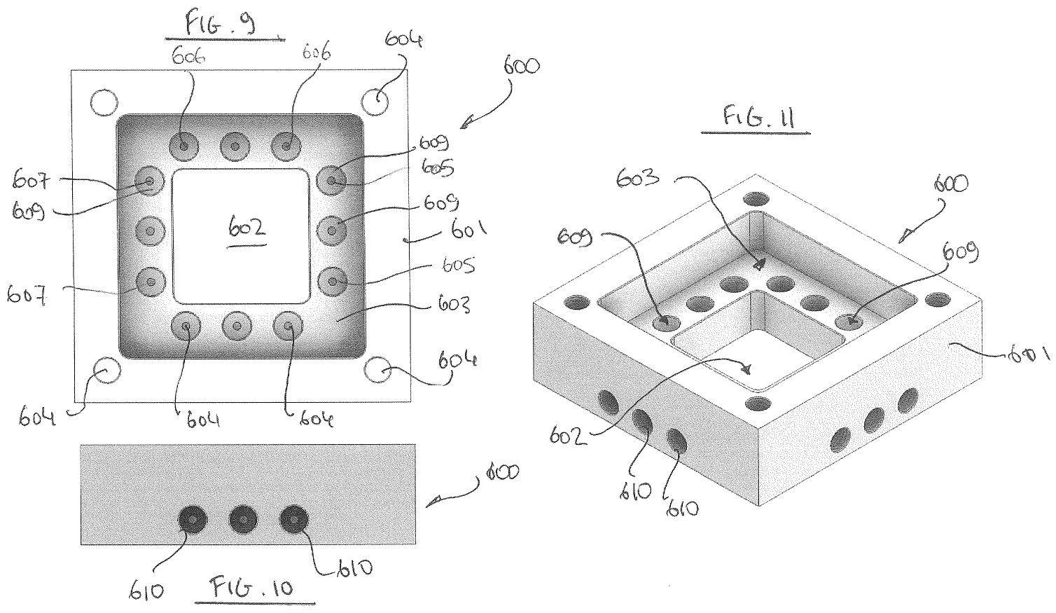

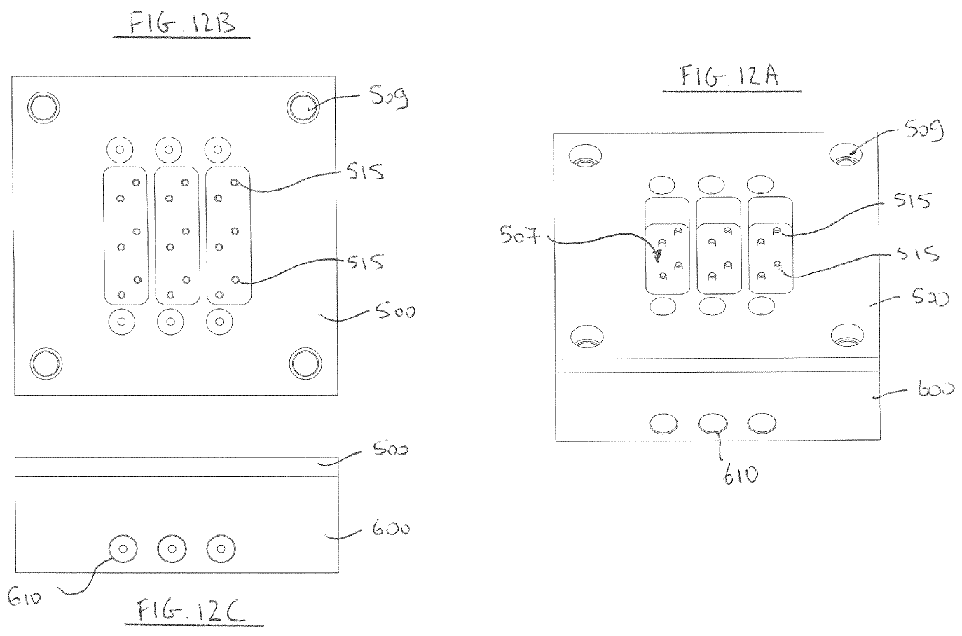

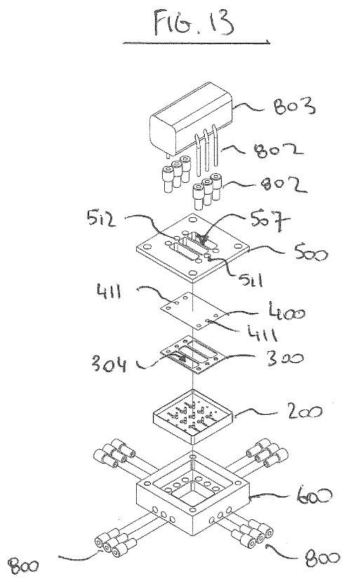

Referring to the drawings, and initially to FIGS. 1 to 11, there is illustrated an assay plate system according to one embodiment of the invention, indicated generally by the reference numeral 100, and individual parts of the assay plate system. The assay plate system 100 comprises an assay plate 200, spacer plate 300, flexible film 400, cover plate 500, and base plate 600 which attach together to form the assembled assay plate system shown in FIG. 12.

In more detail and referring to FIGS. 3 to 5, the assay plate 200 comprises a square planar plate with an upper surface 201, lower surface 202, and a peripheral sidewall 203 that extends around the upper surface 201 of the plate. The plate is machined from acrylic but may also be may also be made by injection moulding using polystyrene, polypropylene or cyclo-olefins. The upper surface 201 has a 3×3 grid array of wells comprising three rows of three wells each 204, 205, 206, a fluid inlet aperture 207 of a fluid inlet conduit disposed at a first end of each row of wells, and a fluid outlet aperture 209 of a fluid outlet conduit disposed at a second end of each row of wells. The fluid inlet conduits are not shown but extend through the plate orthogonally to the upper surface of the plate and function to provide fluid from a fluidic conduit in the base plate to the fluid inlet apertures 207 on the upper surface of the plate. Likewise, the fluid outlet conduits are not shown but also extend through the plate orthogonally to the upper surface of the plate and function to receive fluid from the fluid outlet apertures 209 and deliver the fluid to a fluidic conduit in the base plate. The fluidic conduits of the base plate are described in more detail below.

The plate also includes additional fluid inlet conduits (not shown), each providing fluid communication between a fluid inlet aperture 211 on the upper surface of the plate and a fluidic conduit in the base plate, and additional fluid outlet conduits (not shown), each providing fluid communication between a fluid outlet aperture 213 on the upper surface of the plate and a fluidic conduit in the base plate. The fluid inlet aperture 211 and fluid outlet aperture 213 are disposed at each end of each column of wells. The arrangement of inlets and outlets, combined with the spacer plate, allows fluidic conduits to be generated along an x or y axis of the plate, as will be explained in more detail below.

Referring to FIG. 5, the supper surface 201 of the assay plate comprises two projections 215 that are spaced around each well by about 90°. The first projection is positioned between the fluid inlet aperture 207 and fluid outlet aperture 209, and the second projection is positioned between the fluid inlet aperture 211 and fluid outlet aperture 213.

Referring to FIG. 4, fluid inlet conduits in the assay plate include a proximal extension section 220 that extends from the lower surface 202 of the plate, and fluid outlet conduits include a proximal extension section 221 that extends from the lower surface 202 of the plate.

Referring to FIGS. 1 and 2, the spacer plate 300 comprises a frame 301 having four sidewalls 302 and two divider walls 303 that together define three rectangular apertures 304 of equal size. In use the frame sits on the upper surface 201 of the assay plate and each aperture 304 partly overlies the wells and inlet and outlet apertures of an assay well system. When the flexible film is placed on top of the spacer plate 300, three fluidic conduits 700 are formed on the top of the assay plate, one for each assay well system. The flexible film is formed of polyurethane, but any liquid impermeable and gas permeable flexible film may be employed, for example a silicone membrane or film.

In more detail and referring to FIGS. 6 to 8, the cover plate 500 is described in more detail. The purpose of the cover plate is to hold the flexible film 400 in position and define the periphery of each fluidic conduit 700. The cover plate 500 comprises a plate body 501 with an upper surface 502, a lower surface 503, and a frame element 504 projecting proud of the lower surface 503. The frame element 504 has a shape that is similar to the frame 301 of the spacer plate 300, with sidewalls 505 and divider walls 506 defining apertures 507 that extend through the plate. Through holes 509 are formed in each corner of the plate body 501 and function to secure the cover plate 500 to the base plate 600 when the assay plate system 100 is assembled. The cover plate 500 and base plate 600 may be formed from PEEK (polyetheretherketone, thermoplastic) as it has good chemical resistance and is autoclavable, or from metal, for example stainless steel, for example Stainless steel grade 17-4.

Referring to FIGS. 9 to 11, the base plate 600 is described in more detail. The purpose of the base plate 600 is to receive and hold the disposable assay plate and provide fluidic architecture for providing a supply of fluid to the assay plate when the system is assembled. The base plate comprises a base plate body 601 having a central aperture 602 with a recessed shoulder section 603. The recessed shoulder section 603 is dimensioned to receive the assay plate 200 in a closely fitting arrangement configured to securely hold the assay plate and allow it to be removed during an assay or when an assay has been completed. An upper periphery of the base plate body 601 comprises four apertures 608 for receipt of fixing screws for fixing the cover plate 500 to the base plate to sandwich the assay plate 200, spacer plate 300 and flexible film 400 there between. A lower part of the base plate body 601 comprises first fluidic conduits 604 and 605 configured to supply liquid to fluid inlet conduits of the assay plate 200, and second fluidic conduits 606 and 607 configured to receive liquid from the fluid outlet conduits of the assay plate 200. Each of the conduits 604 to 607 is L-shaped and has a first opening 609 disposed on the recessed shoulder section 603 configured to receive the proximal extension sections 202, 221 in a water-tight manner and a second openings 610 disposed in a sidewall of the base plate body 601 configured to fluidically connected with fluid supply and fluid drainage conduits. The base plate 600 is formed from PEEK (polyetheretherketone, thermoplastic) as it has good chemical resistance and is autoclavable.

In use, and referring to FIGS. 1 and 12, the assay plate system 100 is assembled by placing a 3-D culture model into each of the wells of the assay plate 200 along with assay fluid, and the assay plate is then inserted into the recessed shoulder of the base plate 600. The spacer plate 300 is then placed on the upper surface of the assay plate 200 such that the apertures 304 of the spacer plate are in register with each assay well system. The flexible film 400 is then placed on top of the spacer plate 300 forming three conduits 700, each conduit 700 being defined by the upper surface of the assay plate 200, the frame 301 of the spacer plate and the flexible film 400. The two projections 215 disposed adjacent each well serve as an additional spacing means to ensure that the film is spaced from the upper surface of the assay plate in each fluidic conduit especially in the regions over the wells.

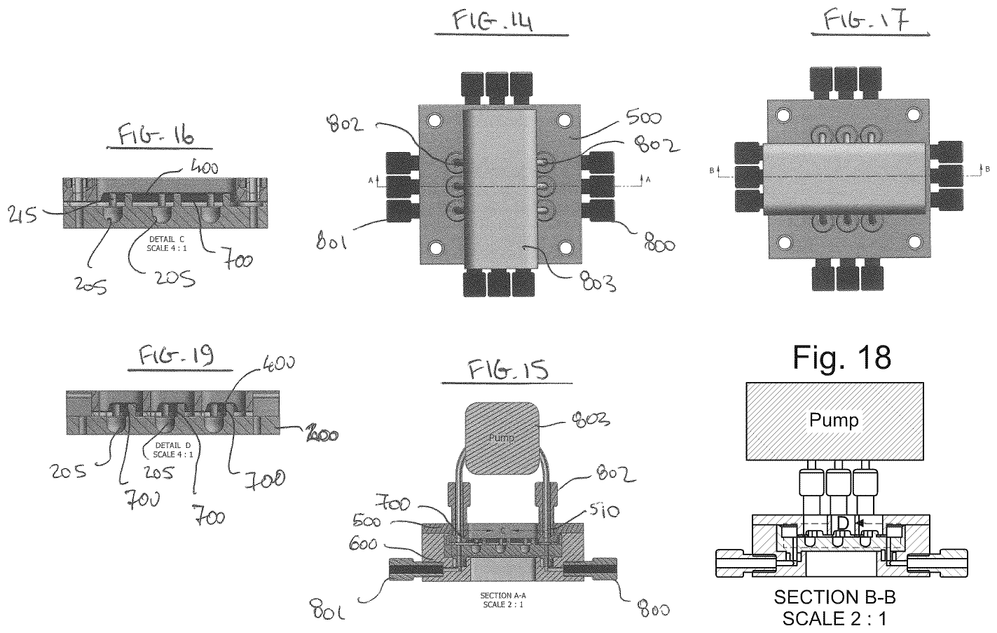

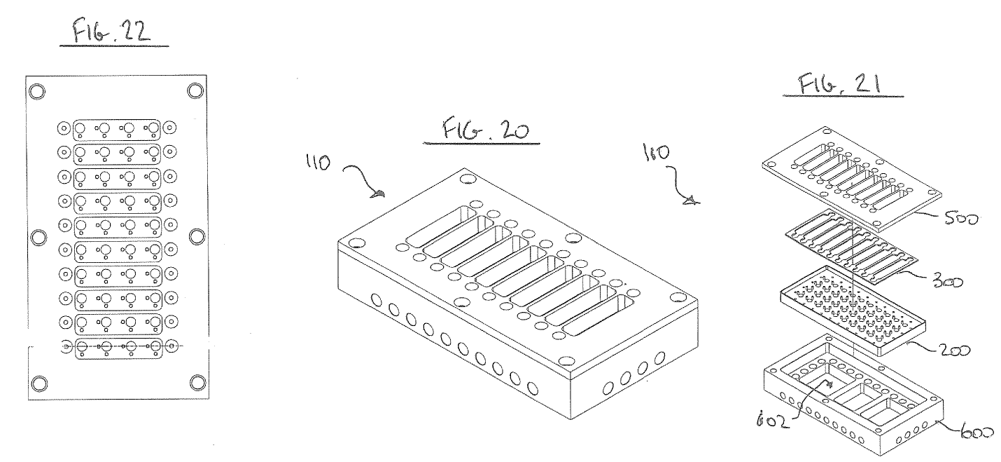

Referring to FIGS. 13 to 19, an assay plate system according to FIGS. 1 to 12 is illustrated. In these figures the assay plate system includes fluid supply, removal and recirculation architecture, specifically including assay fluid supply conduits 800, assay fluid removal conduits 801, and fluid recirculation system comprising conduits 802 and a pump 803. In this embodiment, the cover plate 500 includes apertures 511 and 512 disposed at each end of each aperture 507 configured to connect with conduits 802 forming a fluid recirculation closed circuit including pump 803. The flexible film 400 in this embodiment includes apertures 411 (see FIG. 13) at each of each fluidic conduit 700 providing fluidic communication between the conduits and the fluid recirculation system. The sectional view of FIGS. 15 and 16 illustrate a fluidic conduit 700 of the assay plate system and the sectional view of FIGS. 18 and 19 illustrate the three fluidic conduits 700. Referring to FIG. 13, the orientation of the spacer plate 300 and cover plate 500 means that the fluidic conduits 700 in the plate extend in the direction of the apertures 304.

In use, the assay plate system of FIGS. 13 to 19 may be used to assay three organoids, for example a heart, lung and liver organoid. Each assay well system may include a sample of each of these three types of organoids, and the three assay well system allow the organoids to be assayed in triplicate. The organoid samples are first placed into a well of the assay plate along with a buffer (or grown in-situ in the plate), and the assay plate is then inserted into the recessed shoulder of the base plate. The spacer plate is then placed on top of the upper surface of the assay plate to partly define the fluidic conduits, and the cover plate is then secured to the base with the cover plate apertures in register with the apertures of the spacer plate to fasten the assembly. Assay fluid supply and removal conduits are connected to the openings 610 is the sidewall of the base plate and are used to add assay fluid to the wells at the start of an assay and to remove and replenish assay fluid during the assay as the need arises. A fluid recirculation system comprising conduits 802 and pump 803 is provided to recirculate fluid across the rows of wells allowing assay fluid of one well in a row of wells to be added to other wells in the row of wells. This allows the assay to determine not only the direct effect of a drug on different organs, but also to determine if the effects of a drug on a heart will have an effect on the functioning of another organ such as the liver or kidney.

Referring to FIGS. 20 to 22, an alternative embodiment of the assay plate system of the invention is illustrated in which parts identified with reference to the previous embodiments are assigned the same reference numerals. In this embodiment, the assay plate system 110 is configured to have ten assay well systems, each assay well system having four wells, and ten fluidic conduits when the assay plate system is assembled.

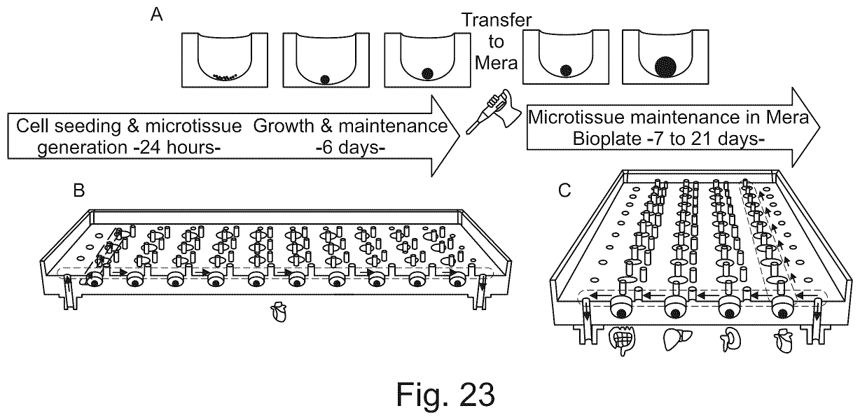

Referring to FIG. 23, the system of the invention and its use is illustrated in the context of a four-organ system. The organs of interest in the human body can be modelled by specific microtissues while microfluidic perfusion is used to model fluid circulation in the human body. The assay plate is arranged in a rectangular format consisting of a 10 row by 4 column well grid into which microtissues can be added or grown from cells. Up to 40 microtissues can be cultured on the plate where rows of wells are fluidically interconnected. Up to four different organ types can be accommodated in the plate as per user requirement (e.g. gastrointestinal, liver, kidney and heart as shown in FIG. 23) with each row of ten housing a different microtissues type. Flow can be directed across columns of a common tissue type (Y-direction) for maturation or maintenance or alternatively flow can be switched to direct flow along a row of four different tissue types (X-direction) thereby initiating recirculation flow to facilitate interorgan communication.

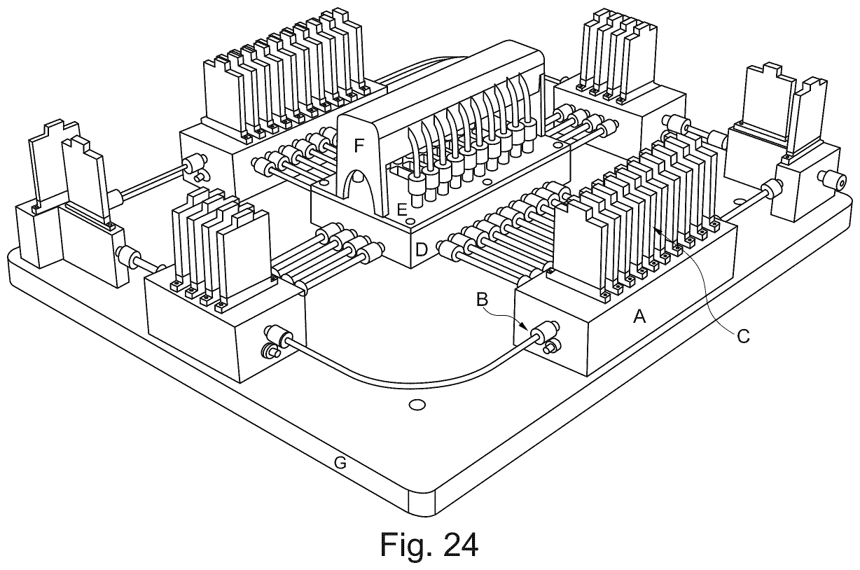

Referring to FIG. 24, an assay plate system of the invention is illustrated and comprises a support plate G, a plurality of valve block manifolds A, a well perfusion control valves module C associated with each valve block manifold, and fluidic conduits fluidically connecting the valve block manifolds in a fluidic circuit. The system also has a recirculation pump F configured to recirculate fluid across the rows of wells.

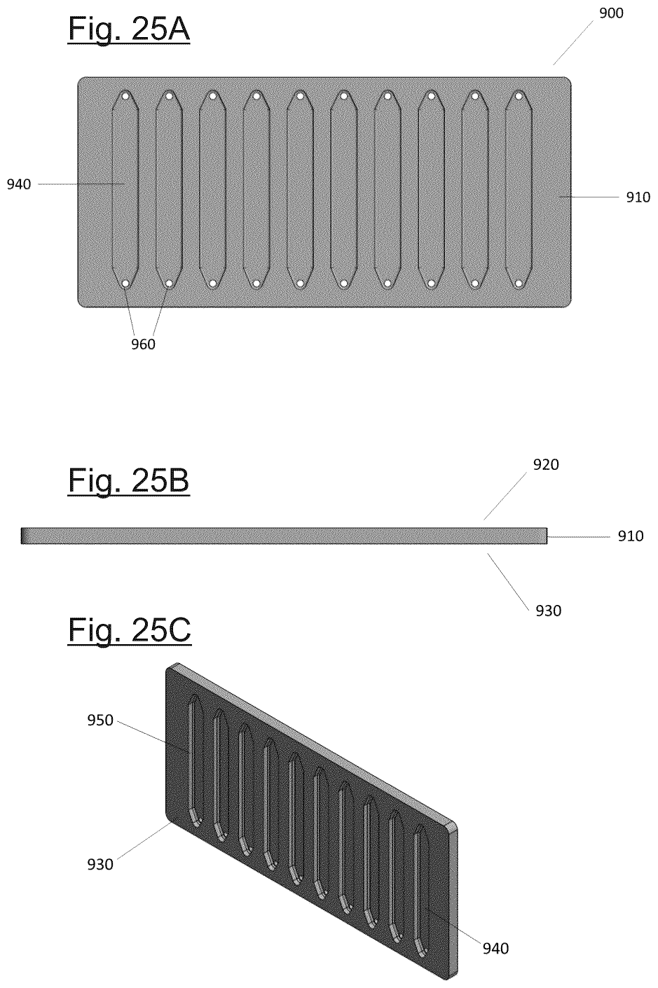

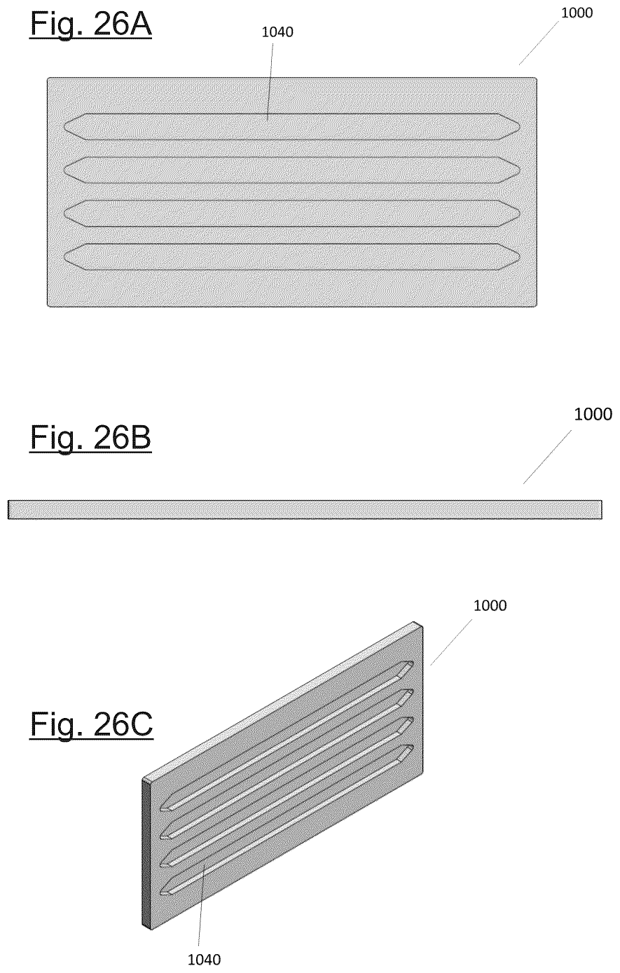

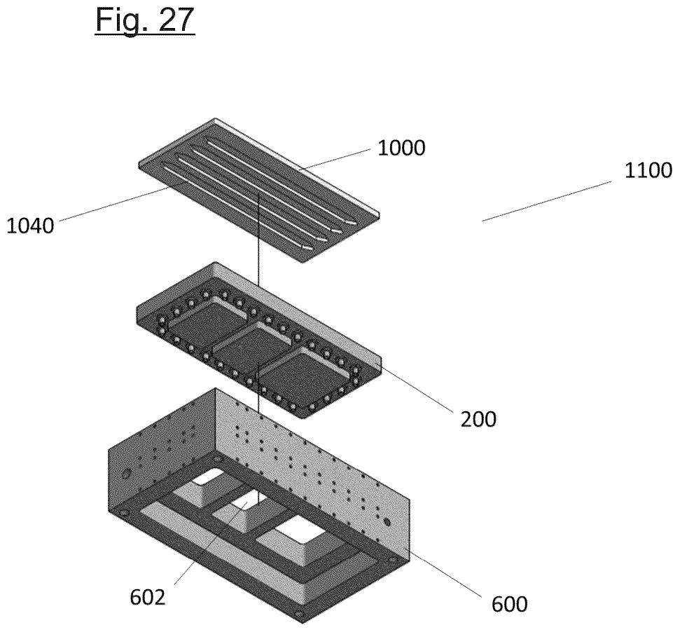

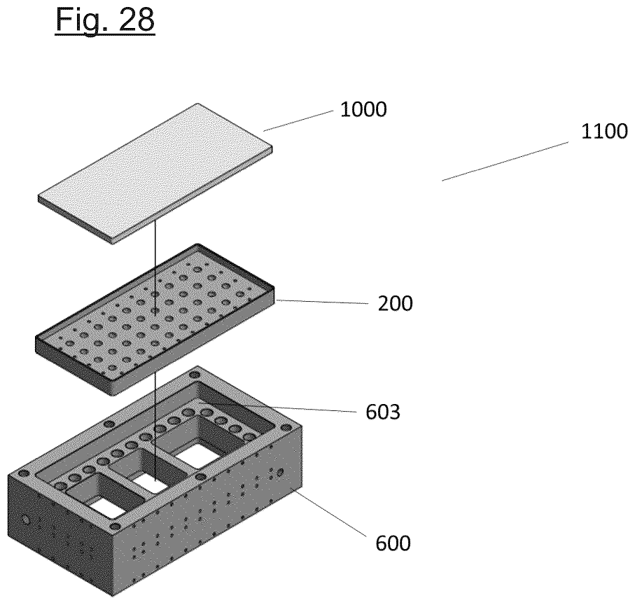

FIGS. 25 and 26 illustrate alternative gasket plates forming part of as assay plate system of the invention. The gasket plates replace the flexible film and spacer plate (and projections) of the previous embodiments. FIGS. 27 and 28 illustrate an assay plate system of the invention comprising the gasket plate of FIG. 25—apart from the gasket plate, the rest of the assay plate system is substantially the same as described above with reference to the previous embodiments. The assay plate 200 has a 10×4 grid array of wells (10 columns and 4 rows). The gasket plate of FIG. 26 is configured to abut the upper surface of the assay plate and form four conduits longitudinally along the top of the assay plate, each conduit containing a column of ten wells. This gasket plate is used during the maturation and growth of the cells or microtissue. The gasket plate of FIG. 25 is configured to abut the assay plate and form ten conduits laterally across the top of the assay plate, each conduit having a row of four wells. This gasket plate is used during the assay stage of the method.

Referring specifically to FIG. 25, the first gasket, indicated generally by the reference numeral 900, is formed from a rectangular Teflon plate 910 machined to a thickness of about 3.6 mm mm, a length of about 114 mm and width of about 54 mm and having a top surface 920 and a bottom surface 930. Referring to FIG. 26A, which is an underneath plan view, a series of parallel transverse recesses 940 are machined on the bottom surface 930 to a depth of about 2 mm providing recess top walls 950 having a thickness of about 1.6 mm, which is sufficiently thin to allow diffusion of gasses through the recess top walls. The recesses have a length of about 46 mm and a width (at their mid-point) of about 6 mm. the spacing between each recess is about 4 mm. Apertures 960 are provided at each end of the recesses 940 providing through holes through the gasket. In use, these through holes align with corresponding through holes in the cover plate providing fluid communication in the assembled assay plate to a fluid recirculation conduit/system disposed above the assay plate that functions to recirculate fluid in a closed loop across rows of wells during an assay phase of the method of the invention. A fluid recirculation conduit/system is illustrated in FIG. 15.

Referring specifically to FIG. 26, the second gasket, indicated generally by the reference numeral 1000, is substantially similar to the first gasket 900 with the exception that the gasket plate comprises a series of parallel longitudinal recesses 1040 instead of transverse recesses 940.

FIGS. 27 and 28 are exploded perspective views from below (FIG. 27) and from above (FIG. 28) of an assay plate system of the invention, in which parts described with reference to the previous embodiments are assigned the same reference numerals. In this embodiment, the assay plate system, indicated generally by the reference numeral 1100, comprises a base plate 600, an assay plate 200, and the second gasket 1000 of FIG. 26. The assay plate 200 is dimensioned to nest in the recessed shoulder of the base plate, and the gasket 1000 is dimensioned to abut the top surface of the assay plate forming four elongate longitudinal conduits, each containing a column of ten wells. A cover plate (not shown) is attached to the base plate to sandwich the assay plate and gasket together.

In use, stem cells are plated into each well of the assay plate, and then the assay plate system is assembled using the gasket plate 1000. A fluid is then passed along each longitudinal conduit to differentiate and mature the cells. The fluid passed along the first conduit may comprise a chemical configured to cause the stem cells to differentiate into a heart microtissue. The fluid passed along the second conduit may comprise a chemical configured to cause the stem cells to differentiate into a liver microtissue. The fluid passed along the third conduit may comprise a chemical configured to cause the stem cells to differentiate into a kidney microtissue. The fluid passed along the fourth conduit may comprise a chemical configured to cause the stem cells to differentiate into a lung microtissue. These fluids cause the stem cells to differentiate into different cell types and to mature. The fluidic system for these fluids is not a closed-loop system and the fluids are provided from reservoirs, through inlet conduits in the base plate and passed slowly along the column of wells whereupon the fluid exits the assay plate through the outlet conduits in the base plate.

Once the four different microtissues have matured, the assay plate is partially disassembled, the gasket 1000 is removed and replaced with the gasket 900, and the assay plate system is reassembled providing ten transverse conduits, each transverse conduit having four wells containing a heart microtissue (first well), a liver microtissue (second well), a kidney microtissue (third well), and a lung microtissue (fourth well). An assay fluid is then pumped through the fluidic conduits of the base plate and the fluid inlet conduits of the assay plate to perfuse the microtissues in the rows of wells. This fluid exits through the fluid outlet conduits of the assay plate and the fluidic conduits of the base plate and is not recirculated. The assay plate system is configured to provide a different fluid to each row of wells. For example, a first row of wells may be provided with a fluid containing a first concentration of a first test molecule, a second row of wells may be provided with a fluid containing a second concentration of the first test molecule, a third row of wells may be provided with a fluid containing a first concentration of a second test molecule, and a fourth row of wells may be provided with a fluid containing a second concentration of the second test molecule, This fluidic system is then closed, and the fluid recirculation system (illustrated in FIG. 15) for the ten conduits is actuated to recirculate fluid across the rows of wells to assay the effect that metabolites produced by one microtissue type has on another microtissue type.

Perfusion control valves

The 4.5 mm width of the thirty-two 24V 2/2 Flipper-Solenoid Valves (00226664, Burkert, Ingelfingen, Germany) allowed the channels to be placed closer together than larger alternatives, whilst also providing the basis for the Ø1.4 mm internal fluidic channels (max diameter tolerated by the valve specifications). The thirty-two valves comprised of two entry valves, two exit valves and twenty-eight inner directional valves, which facilitated the control of the direction of fluid movement.

### Valve Block Manifolds

Each valve block (Acrylic, Engineering Steels, Limerick, Ireland) can hold two Burkert valves; there is a male and female block to complete the valve block manifold assembly, into which ¼-28″ IDEX fittings (CIL-XP-301X, Darwin Microfluidics, Paris, France) are connected. The fluidic seal between the individual blocks within the assembly is maintained with O-rings to tolerance as per the radial compression design recommendations. As an added precaution, an m3 threaded rod intersects the entire valve block manifold. An m3 nut on the distal ends of the rod provides a compressive force to complement the fluidic sealing capacity of the O-rings.

To minimise the introduction of air into the system, an inlet/outlet T-block (PMMA, Engineering Steels, Limerick, Ireland) was designed to omit tubing from the design whilst maintaining the functionality of a directional switch valve. Three ¼-28″ IDEX fittings (CIL-XP-301X, Darwin Microfluidics) are threaded into the block to complete the connection to the system.

The entire system is housed on a block of Aluminium (Engineering Steels Limerick, Ireland). This allows parts to be fastened in place, providing a neat solution and consistency during experiments by reducing moving parts. The assembly of the Mera system is depicted in FIG. 24.

### Perfusion Control

Two peristaltic pumps (KCM-B168, Kamoer Fluid Tech, Shanghai, China) are used to control perfusion through the system at a constant rate of approximately 2.5 ml/min currently, running for a minute across each line once per hour. This rate can be adapted depending on the individual needs of the cell model. A third pump (Takasago Electric Inc, Nagoya, Japan) F used for recirculating media is in the validation stage. The recirculation line is approximately 300 mm long. The recirculation pump runs at the same rate as the peristaltic pumps, ensuring a consistent environment for microtissues. The system is being tested to replicate current results while diminishing the requirement for media from an external source. The fluidic lines consist of PTFE tubing (⅛″ OD, 1/16″ ID) (BL-PTFE-3216-20, Darwin Microfluidics, Paris, France).

### Imaging System

A custom epi-florescence microscope was designed in-house and incorporated within the environmental chamber for automated in-situ imaging of microtissues. The optical assembly of the imaging module consists of a 470 nm blue colour (M470L5, Thorlabs, Germany) and a 530 nm green colour (M530L4, Thorlabs, Germany), an LED light source for excitation of the sample, a filter cube (excitation filter, emission filter and dichroic mirror), a camera tube (WFA100, Thorlabs, Germany) and an objective lens with 5× magnification (46143, Mitutoyo, Japan). A CMOS camera (acA1920-155ucMED, Basler AG, Germany) was used for high-resolution microscopic imaging. The microscope was integrated with an automated 3-axis motion control system. A python-based imaging GUI was developed using a Basler Pylon library to acquire the images and automatically control the motion of the microscope.

For reproducibility, brightfield and fluorescence images were acquired for all microtissue data using an IX70 Olympus inverted microscope (Olympus, Tokyo, Japan) with a 4×objective and an Am Scope Microscope Digital Camera (MU1003, AmScope, UK). All images were analysed using the Z-stacker App (CAPPA-CIT, Cork, Ireland) which allows for contract/intensity-based image analysis for user selectable regions of interest. It was developed using OpenCV (Open-source Computer Vision library), a library of programming functions aimed mainly at machine vision and image analysis applications and consists of the following process, image alignment, sharpness map calculation, map comparison and image composition.

### Environmental Chamber

To grow and maintain reproducible microtissues, mammalian cells require a temperature of 37° C. and 5% CO2 within a sterile environment. To achieve this, an environmental chamber with a dimension of 1.5 m×1 m×2 m was fabricated using stainless steel (SQ Fabs. Limerick, Ireland). The chamber was designed to be easily accessible for Bioplate removal, maintenance, and sterilization procedures. A temperature control system consisting of a digital temperature sensor (DS18B20, DFRobot, China) and compact enclosure heater fans (02801.1-01, STEGO, Germany) were integrated to maintain a temperature of 37° C. while a 12V DC axial fan was mounted on top of the enclosure heater fans to increase airflow velocity. A laminar flow system consisting of a HEPA filter and a centrifugal fan is built into the environmental chamber.

CO2 was supplied in a 6 kg VB canister (BOC, Limerick, Ireland) which was safety tethered adjacent to the unit. CO2 sensor output is required to control the activation of the CO2 system. A high concentration, low power CO2 sensor (2091-EXPLORIR-WHV-E-100-ND, Gas sensing Solutions Ltd, UK) and a two-port solenoid valve (VX230AG, SMC corporation) are used to control the inflow of the CO2 gas to the environmental chamber.

Reagents including media and buffers were stored in a miniature fridge which was retrofitted to include a length of tubing to supply fluids to the system, the length of tubing is elongated to include sufficient slack to ensure the fluid rises to 37° C. via radiant heat within the hour timeframe between doses.

Various parameters of a cell may be determined as part of the assay, for example cell health as determined by oxygen consumption, or pH or redox parameters. Generally, this involves using an assay fluid comprising, as an example, an oxygen sensitive fluorescent dye and monitoring a fluorescent signal being emitted from the transparent base of the well using a suitable imaging device.

In this regard, the invention also provides an assay rig comprising a frame, a holder for an assay plate system of the invention attached to the frame, an imaging device attached to the frame and disposed underneath the assay plate system when the assay plate system is mounted to the holder.

In any embodiment, the rig comprises a coupling device for coupling the imaging device to the frame, wherein the coupling device is configured to move the imaging device relative to the holder so as to allow different wells of the assay plate to be imaged.

In any embodiment, the rig comprises a processor operatively coupled to the coupling device and the imaging device and configured to generate images of specific wells in response to input instructions.

In any embodiment, the rig comprises a fluid tight container in which the holder and imaging device are contained within the fluid tight container.

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

### REFERENCE NUMERALS

Assay plate system 100
Assay plate 200
Spacer plate 300
Flexible film 400,
Cover plate 500
Base plate 600
Upper surface of plate 201
Lower surface of plate 202
Peripheral sidewall of plate 203
Wells 204, 205, 206
Fluid inlet aperture (rows) 207
Fluid outlet aperture (rows) 209
Fluid inlet aperture (columns) 211
Fluid outlet aperture (columns) 213
Projections 215
Extension sections of fluid inlet conduits 220
Extension sections of fluid outlet conduits 221
Spacer plate frame 301
Spacer plate sidewalls 302
Spacer plate divider walls 303
Spacer plate rectangular apertures 304
Flexible film apertures 411
Cover plate body 501
Cover plate upper surface 502
Cover plate lower surface 503
Cover plate frame element 504
Cover plate sidewalls 505
Cover plate divider walls 506
Cover plate apertures 507
Cover plate through holes 509
Cover plate fluid recirculation conduits 510
Cover plate apertures 511, 512
Base plate body 601
Base plate central aperture 602
Base plate recessed shoulder section 603.
Base plate fixing screw apertures 608
Base plate first fluidic conduits 604, 605
Base plate second fluidic conduits 606, 607
Base plate fluidic conduit first opening 609
Base plate fluidic conduit second openings 610
Conduits 700
Fluid recirculation conduits 802
Fluid recirculation pump 803
First gasket 900
Gasket plate 910
Top surface of gasket plate 920
Bottom surface of gasket plate 930
Transverse recesses 940
Recess top walls 950
Apertures 960
Second gasket 1000
Longitudinal recesses 1040
The assay plate system 1100

1. An assay plate system for assaying a cell culture model, the assay plate system comprising:
an assay plate comprising an upper surface and comprising a grid array of wells comprising:
at least two rows of wells, each row comprising at least two wells, each well having an open top formed in the upper surface and a closed transparent base;
at least two columns of wells, each column comprising at least three wells, each well having an open top formed in the upper surface and a closed transparent base;
a fluid inlet conduit for each row of wells having a first fluid inlet aperture disposed on the upper surface at a first end of each row of wells; and
a fluid outlet conduit for each row of wells having a first fluid outlet aperture disposed on the upper surface at a second end of each row of wells; and
a first gasket comprising a plate having a top surface, a bottom surface, and a plurality of elongated recesses formed in the bottom surface that together with the upper surface of the assay plate define a fluidic conduit for each row of wells providing fluidic communication between the first fluid inlet aperture, the row of wells and the first fluid outlet aperture in series, in which the first gasket is formed from Teflon and wherein a top wall of each recess is gas permeable.

2. (canceled)

3. The assay plate system according to claim 1, in which the grid array of wells has a same number of columns and rows of wells.

4. The assay plate system according to claim 1, comprising a second gasket configured for replacement of the first gasket in the assay plate system, the second gasket comprising a plate having a top surface, a bottom surface, and a plurality of elongated recesses formed in the bottom surface that together with the upper surface of the assay plate define a fluidic conduit for each column of wells providing fluidic communication between the column of wells.

5. The assay plate system according to claim 1, in which the assay plate comprises:
a fluid inlet conduit for each column of wells having a first fluid inlet aperture disposed on the upper surface at a first end of each column of wells; and
a fluid outlet conduit for each column of wells having a first fluid outlet aperture disposed on the upper surface at a second end of each column of wells.

6. The assay plate system according to claim 1, in which:
the fluid inlet conduit for each row or column of wells comprises a second fluid inlet aperture disposed on a lower surface of the assay plate at the first end of each row or column of wells; and
the fluid outlet conduit for each row or column of wells comprise a second fluid outlet aperture disposed on a lower surface of the assay plate at the second end of each row or column of wells.

7. The assay plate system according to claim 6, including a base plate comprising:
a central through aperture comprising a recessed shoulder that extends around a periphery of the central through aperture, in which the recessed shoulder is dimensioned to receive the assay plate;
first fluidic conduits configured to fluidically connect with the second fluid inlet apertures of the assay plate when the assay plate is mounted on the recessed shoulder of the base plate; and
second fluidic conduits configured to fluidically connect with the second fluid outlet apertures of the assay plate when the assay plate is mounted in the recessed shoulder of the base plate.

8. The assay plate system according to claim 6, in which the fluid inlet conduits and fluid outlet conduits of the assay plate comprise extension sections that extend proud of a lower surface of the assay plate, and in which a recessed shoulder of the base plate comprises sockets dimensioned to receive the extension sections and fluidically connect the fluid inlet conduits and fluid outlet conduits of the assay plate with the with the first fluidic conduits and second fluidic conduits of the base plate, respectively.

9. The assay plate system according to claim 7, which each first fluidic conduit (604) and second fluidic conduit of the base plate comprises an outlet/inlet aperture disposed on an external sidewall of the base plate.

10. The assay plate system according to claim 7, in which a upper surface of the base plate is configured for coupling to a cover plate such that when the base plate and cover plate are coupled together with the assay plate and gasket sandwiched between the base plate and cover plate, the cover plate abuts the top surface of the gasket plate.

11. The assay plate system according to claim 1, comprising a first fluid supply system to supply fluid to at least a first row of wells, the first fluid supply system comprising a first fluidic supply inlet conduit fluidically connected to an inlet aperture of a first fluidic conduit of the base plate, a first fluidic supply outlet conduit fluidically connected to an outlet aperture of a second fluidic conduit of the base plate, and a pump operable to pump fluid along the first row of wells.

12. The assay plate system according to claim 11, comprising a dedicated first fluid supply system for each row of wells.

13. The assay plate system according to claim 1, comprising a second fluid supply system to supply fluid to at least a first column of wells, the second fluid supply system comprising a second fluidic supply inlet conduit fluidically connected to an inlet aperture of a first fluidic conduit of the base plate, a second fluidic supply outlet conduit fluidically connected to an outlet aperture of a second fluidic conduit of the base plate, and a pump operable to pump fluid along the first column of wells.

14. The assay plate system according to claim 11, comprising a dedicated second fluid supply system for each row of wells.

15. The assay plate system according to claim 1, in which at least one of the elongated recesses in the first gasket comprises an aperture formed at each end of the elongated recess, and a cover plate comprises corresponding apertures configured to register with the apertures in the gasket when the assay plate system is assembled.

16. The assay plate system according to claim 15, including a fluid recirculation system to recirculate fluid across wells in a row of wells, comprising a fluid recirculation conduit having a first end in fluid communication with a first aperture in the cover plate and a second end in fluid communication with an opposed second aperture in the cover plate, and a pump operable to pump fluid along the fluid recirculation conduit.

17. A method of assaying a microtissue that employs an assembled assay plate system, the method comprising:
providing a first microtissue sample in a first well of a first row of wells;
providing a second microtissue sample in a second well of the first row of wells;
optionally, providing a third microtissue sample in a third well of the first row of wells;
delivering a first fluid to a fluid inlet conduit of the first row of wells so that the first fluid is incubated with each microtissue sample in the first row of wells; and
imaging the wells through a transparent base of each well with an imaging device.

18-26. (canceled)
